(19) United States
(12) Patent Application Publication
(10) Pub. No.: US 2011/0196302 A1
(43) Pub. Date: Aug. 11, 2011

(54) Cannula apparatus

(76) Inventors: Michael R. Gildersleeve, Sharon, MA (US); George G. Brusard, Lowell, MA (US); John J. Landy, III, Billerica, MA (US)

(21) Appl. No.: 12/658,754

(22) Filed: Feb. 6, 2010

(51) Int. Cl. A61M 25/00; A61B 1/32; A61M 1/00

(52) U.S. Cl. 604/151; 604/523; 600/205

(57) ABSTRACT

A canister is fabricated with a tubular wall and with end caps at respective ends of the tubular wall to form an enclosed cannula. Perforations, or openings, extend through at least the tubular wall and one end cap of the canister to facilitate fluid flow into, through and out from the canister to facilitate use of the cannula for irrigation (lavage) and aspiration (suctioning) procedures A fluid port provides an entry and exit for fluid passing into, through and out from the canister. An additional opening is provided to facilitate positioning a thermo probe in the path of fluid flow through the cannula. The openings are preferably substantially rectangular in configuration with rounded corners and are disposed to provide fluid flow openings through approximately 14% of the surface area of the cannula.

### BACKGROUND OF THE INVENTION

1. Field of Application

This invention relates to sterile, single use, cannulae; and, more particularly, to such cannulae for aspiration of fluid(s) from an anatomical body and/or irrigation of fluid(s) through an anatomical body.

2. Description of the Prior Art

The invention involves a sterile, single-use cannula apparatus used for safely suctioning (aspirating) fluids from, and/or effectively delivering fluids into or through, a body cavity such as the peritoneal (abdominal) or pleural (chest) cavity, at the point-of-use, during open, laproscopic, or closed surgical procedures. Applications include, but are not limited to, hyperthermic therapies and warmed fluid lavages.

During surgeries some physicians often require a means of aspirating (suctioning out) fluids (which may include a mixture of blood, crystalloid solutions, saline, chemotherapy solutions, with residual tissue debris and fats) that may collect in a body cavity by using various commercially available cannulae such as those shown and described in U.S. Pat. Nos. 4,014,333; 5,374,244: 5,968,008 and the like.

In the course of suctioning out the aforesaid types of fluids, using cannulae of the kinds shown in the aforementioned patents, if the point source of concentrated vacuum comes in direct contact with tissue or an internal organ, or organs, suction damage to the tissue may occur, increasing the possibility of organ damage which may adversely affect patient recovery and overall prognosis. Furthermore, if the point source of vacuum becomes occluded by clogging an opening or openings in a conventionally available typical cannula, or by impinging on adjacent tissue, or by collapsing on itself as a result of compression from surrounding tissue, suction efficacy may be significantly compromised.

Typical, commercially available and traditional cannula constructions may feature a flexible, wand-like, flaccid body with a relatively limited number of small perforations running along its finger-like, or ribbon-like, body and a means of connecting to an external vacuum source or pump via tubing. The performance of these types of traditional catheter-like cannulae is generally sub-optimal as their geometry and construction are conducive to partial occlusion during use that could reduce suction efficacy. In the event of such a partial occlusion, the vacuum often becomes more concentrated in the openings that are still open, increasing the risk of adverse tissue contact.

Some physicians often also require a means of delivering fluids within the confinements of a body cavity for reasons such as lavaging during the course of a surgical procedure. Often times it is desirable to deliver the fluid by an external pump so that tissue exposure to the fluid is maximized by optimizing fluid distribution and agitation within the body cavity.

Furthermore, in the event conventionally available traditional cannulae are used to deliver fluid, omni-directional flow is generally limited due to the relatively low number of perforations per cannula length and their low total perforation cross-sectional area (<1%).

Due to the flexible, flaccid body of conventionally available, traditional cannulae, increasing the number of perforations and or cross-sectional area of perforations are unattractive options that could compromise cannula body integrity needed to resist collapse under negative pressure or external compression.

It is also often desirable for physicians to know the temperature of fluid that is delivered to the body cavity, or suctioned out from the body cavity, such as during the course of a hyperthermic-type therapy.

Conventionally available, traditional cannulae, such as those shown and described in the above enumerated patents, have no feature to secure a temperature probe within the fluid path. This can lead to temperature readings which most likely, reflect only the local fluid temperature in the immediate vicinity of the probe, and not the fluid temperature within the body cavity as a whole.

### SUMMARY OF THE INVENTION

It is therefore an object of this invention to provide a new and novel cannula.

It is another object of this invention to provide new and novel sterile single use, cannulae.

It is another object of this invention to provide new and novel cannulae for aspiration and/or irrigation of fluid(s) from and/or into and through an anatomical body.

It is another object of this invention to provide new and novel cannulae with a relatively greater number of perforations per cannula length and/or surface area.

It is another object of this invention to provide new and novel cannulae with a relatively greater total perforation cross-sectional area.

It is another object of this invention to provide a new and novel canula arrangement which is adapted to mount a temperature probe.

It is yet another object of this invention to provide a new and novel canula arrangement which is adapted to mount a temperature probe in the path of fluid flow through the cannula.

It is yet another object of this invention to provide a new and novel canula arrangement which is adapted to mount a temperature probe in the path of fluid flow through the cannula during aspiration and/or irrigation procedures.

The invention provides a cannula apparatus that is connectable to an external vacuum source to suction fluid from a body cavity and a means of isolating the concentrated source of vacuum at the point-of-use away from adjacent body tissue and organs, thus minimizing the risk of post-operative hematoma formation that may hinder patient recovery of the patient.

The invention further provides a cannula apparatus for effectively distributing fluid omni-directionally within the confines of a body cavity when utilized, in a positive pressure application with an external pump.

The invention also provides a means of sensing fluid temperature ported into or broadcast out of the cannula apparatus body by incorporating a mechanism to secure a temperature probe within the directed fluid path inside the cannula apparatus body.

Other objects of this invention will hereinafter become obvious from the following description of the preferred embodiments of this invention.

### BRIEF DESCRIPTION OF THE DRAWINGS

In the drawings:

FIG. 1 is a perspective drawing of a cannula apparatus, incorporating and embodying the principles of and the instant invention;

FIG. 2 is a perspective drawing of the cannula apparatus of FIG. 1, with associated components attached to facilitate use of the cannula, incorporating and embodying the principles of and the instant invention;

FIG. 3 is a front elevation view of the cannula with components of FIG. 2;

FIG. 4 is a vertical section through the cannula and components of FIGS. 2 and 3 taken on line 4-4 of FIG. 3;

FIG. 5 is a vertical section through the cannula and components of FIGS. 2 and 3 taken on line 5-5 of FIG. 3;

FIG. 6 is a horizontal section through the cannula and components of FIGS. 2 and 3 taken on line 6-6 of FIG. 3;

FIG. 7 is a showing of the circled area marked 7 of FIG. 4, enlarged to better show details thereof;

FIG. 8 is an isometric drawing, looking at the upper detail, of the accessory attaching cap for the cannula of FIGS. 1-5;

FIG. 9 is a vertical drawing of the accessory attaching cap for the cannula of FIGS. 1-5;

FIG. 10 is an isometric drawing, looking at the underside detail, of the accessory attaching cap for the cannula of FIGS. 1-5;

FIG. 11 is an isometric drawing, looking at the upper detail, of an end cap for the cannula of FIGS. 1-5;

FIG. 12 is a vertical section through the cannula and components, similar to FIG. 4, and showing a fluid path there-through as it would be during an aspiration (suction) procedure;

FIG. 13 is a vertical section through the cannula and components, similar to FIG. 4 and showing a fluid path there-through as it would be during an irrigation (lavage) procedure; and

FIG. 14 is a schematic showing of the canulla of FIGS. 1-5, with components as shown in FIGS. 2-5 and 12 and 13 along with other components disposed for use with a patient showing the cannulae, incorporating the instant invention, in use both for an aspiration (suction) procedure and/or an irrigation (lavage) procedure.

### DESCRIPTION OF THE INVENTIVE EMBODIMENT

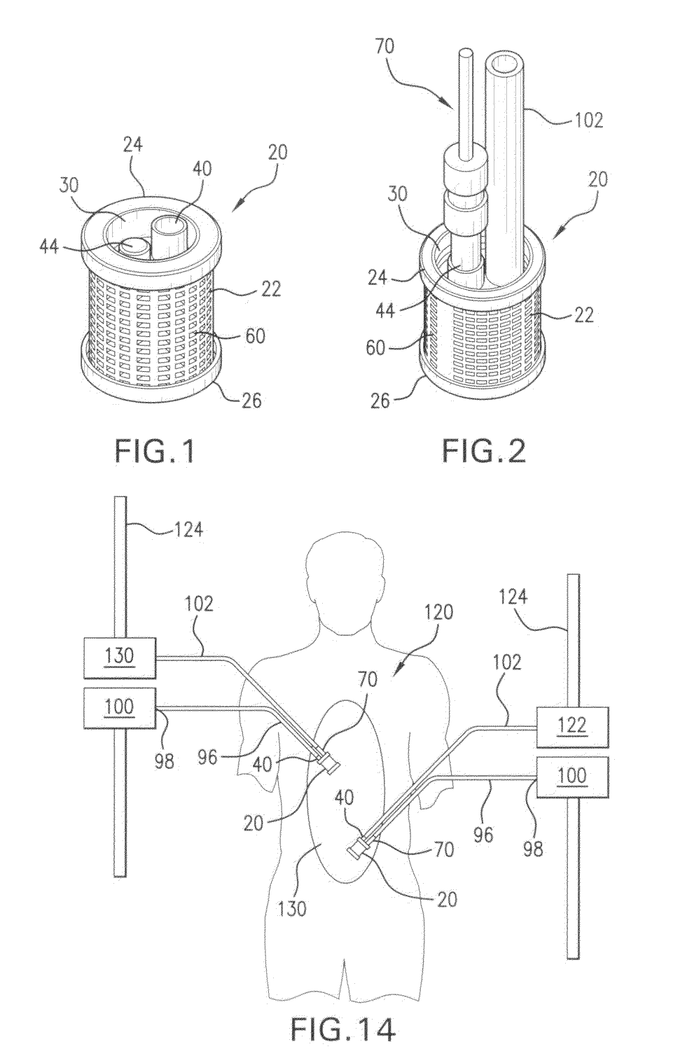

With reference particularly to FIGS. 1-5 there is shown a cannula apparatus or canister 20, incorporating the instant invention, which includes: a tube or body section 22; a component attaching end cap or piece 24 (FIGS. 1-5, and FIGS. 6-10), and an end cap or piece 26 (FIGS. 1-5 and FIG. 11).

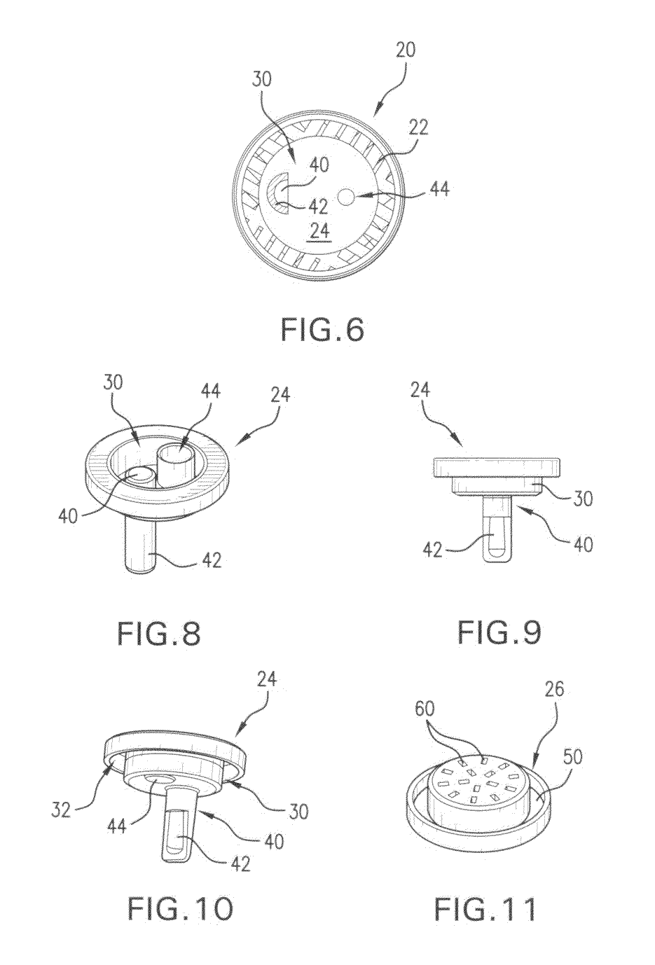

Component attaching end piece 24 is formed with a recessed portion 30 (FIGS. 1, 2, 4, 5, 6, and 8-10) and a circumferential channel 32 (FIGS. 4, 5, 8 and 10) sized and configured to receive a first edge 34 (FIGS. 5 and 7) of tube section 22. A fluid port 40 (FIGS. 1, 4, 6 and 8-10) is carried by and extends through and below recessed portion 30 and includes a fluid directing end portion 42 (FIGS. 6 and 8-10) for purposes to be further described hereinafter. An accessory port 44 (FIGS. 1-2) also extends through and is carried by recessed portion 30 also for purposes to be hereinafter described.

End cap 26 (FIGS. 1-5 and 11) includes a circumferential channel 50 (FIGS. 4, 5 and 11) sized and configured to receive a second edge 52 (FIGS. 4 and 5) of tube section 22.

It should thus be seen and understood that cannula or canister 20 is thus assembled by inserting first edge 34 of tube section 22 into channel 32 of component attaching end piece 24 and second edge 53 of tube section 22 into channel 50 of end cap 26.

Tube section 22, end piece 24 and end piece 26 are preferably fabricated from rigid to semi-rigid, perforated, thermoplastic or the like material. A plurality of perforations or openings 60 (FIGS. 1-5) are provided about and through the entire circumference of tube section 22; with similar perforations or openings provided through end piece or cap 26 (FIG. 11}. End caps 24, 26 are preferably formed of rigid thermoplastic.

Cannula apparatus 20 and its components are constructed from biocompatible thermoplastics, including but not limited to, polyethylene, polypropylene, polyester, nylon, polyvinyl chloride, acrylonitrile butadiene styrene, polytetrafluoroethylene, acrylic, polycarbonate, etc. The thermoplastics utilized are also to be compatible with various sterilization processes, including but not limited to, ethylene oxide gas and gamma-radiation. End pieces 24, 26 may be joined to tube section 22 by a variety of fabrication techniques including, but not limited to, adhesive bonding, solvent bonding, ultrasonic welding, RF welding, direct heat, spin welding, snap interference fit, etc. to form cannula or canister 20.

In the preferred embodiment, perforations, or openings, 60 are oval-like or like rectangles with rounded corners and preferably measure about 0.050″×0.090″ with a percentage open area thru tube section 22 of about 14%. The dimensional envelope of cannula apparatus canister 20 is about 1.50″ H1.25″ diameter. Tube section 22 may be fabricated from rigid to semi-rigid material of about 0.120″ thickness; however, tube section 22 may be thinner or thicker depending on the intended use application for cannula 20.

The perforated material in our commercial embodiment to which you are referring in fact does not come as a flat item. Tube section 22 of canister 20 is preferably fabricated as an extruded tube (with no seams or welds), and having some rigidity and sufficient mechanical strength and integrity to prevent collapse during suctioning. While perforations or openings 60 are selected to be 0.050″×0.090″—and are not perfect circles but more like rounded rectangles or ovals other sizes of perforations or openings may be utilized as long as cannula 20, when in a suctioning procedure does not do damage to body organs in the proximity of the materials and fluids being suctioned. The depth of perforations 60 is dictated by the wall thickness of tube section 22, which in this embodiment is 0.120″. The preferred wall thickness range may be between 0.045″ to 0.155″.

The selected and preferred 14% open area for openings 60 and 62, as well as the nominal opening size of 0.050×0.090, characteristics represent cannister specifications that will be consistent with the inventive embodiment(s) shown and described. The percent open area should be at least about 1%, with a preferred range between about 10% and 40%, and no greater than about 60%. Opening size should be at least about 0.040 in diameter, with a prefered opening size range between about diameters of 0.050 and 0.115, and no greater than about 0.200 in diameter.

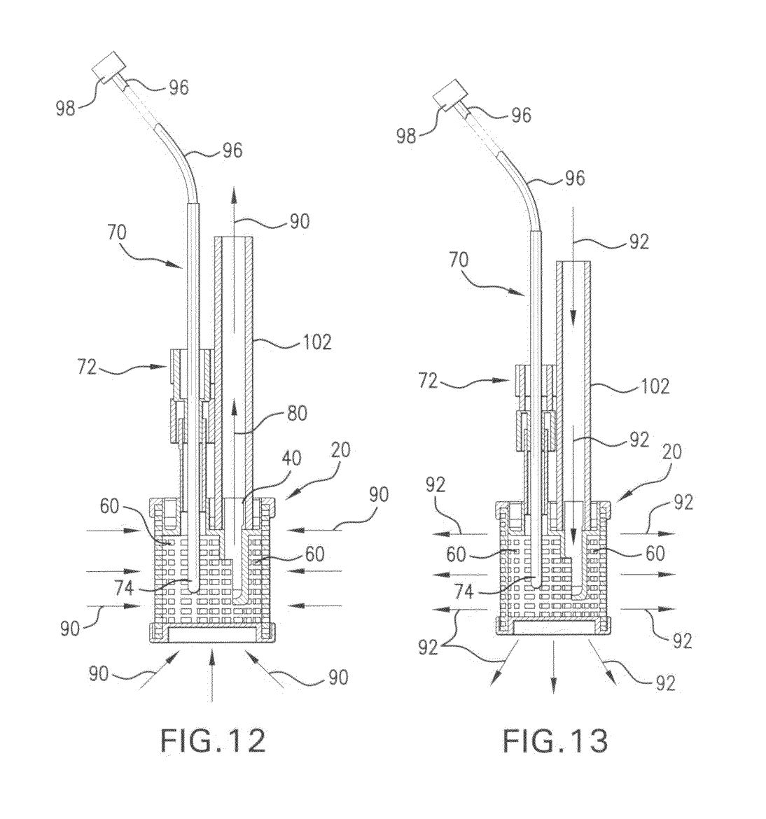

A temperature probe, such as a thermocouple or the like, 70 (FIGS. 2-5, and 12-13) may be secured to accessory end piece 24 by a variety of means including, but not limited to, compression-type fittings such as a Tuohy-Borst adapter 72 or a Swagelok (not shown), or by being adhesively potted in place. When so positioned a lower portion 74 (FIGS. 4, 5, 12 and 13) of probe 70 will be disposed in the path of fluid flow 90 during an aspiration (suction) procedure as shown in FIG. 12 as well as being disposed in the path of fluid flow 92 during an irrigation (lavage) procedure as shown in FIG. 13.

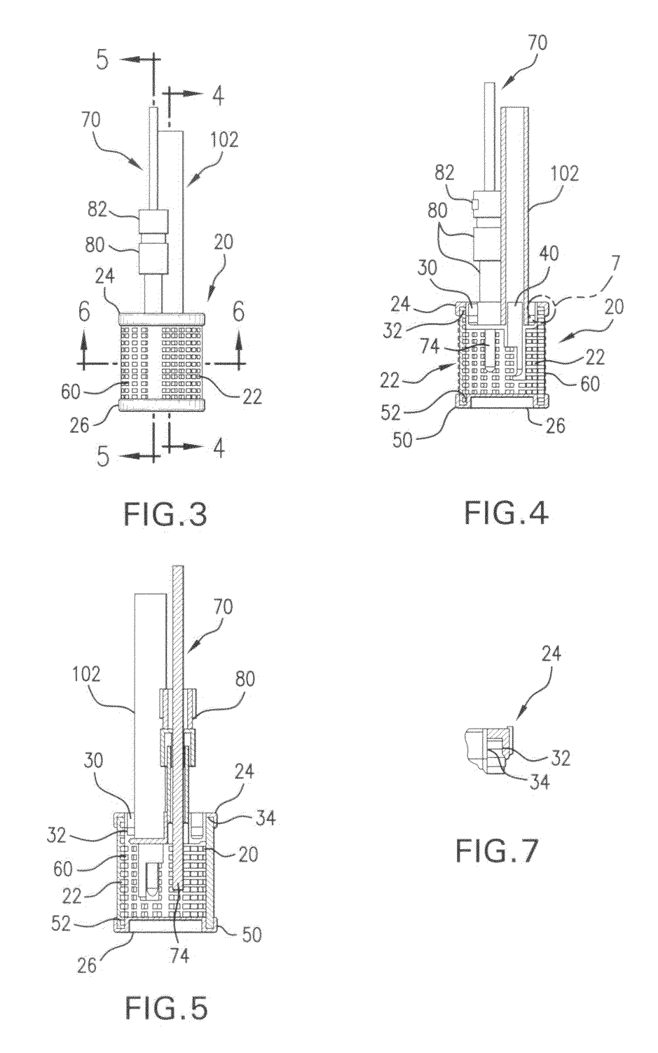

Tuohy-Borst adapter or fitting 72 is essentially comprised of a plastic cylinder 80 (FIGS. 3-5) with a cap 82 that threads onto the outside of cylinder 80 and a flattened elastomeric o-ring gasket 84 (FIG. 5) that is concentric with cylinder 80 through which thermocouple 70 is inserted. Lower portion 74 of thermocouple 70 is inserted through concentric hole 44 (FIG. 10), in cap, 24 that is disposed and sized to accommodate the diametric size and configuration of thermocouple 70. O-ring gasket 84 is seated upon a stationary ledge 86 (FIG. 5) within cylinder 80. Cap 82 has a bushing feature 88 that extends into cylinder 80 such that as it is threaded down, it compresses o-ring gasket.84. As o-ring gasket 84 is compressed, the through hole diameter gets smaller, effecting an interference fit around thermocouple 70. Once cap 82 is threaded down completely, thermocouple 70 is secured within cylinder 80 mechanically. Thermocouple 70 also includes an electric conductor 96 (FIGS. 12 and 13) that is provided with a connector 98 to interconnect thermocouple 70 with a conventionally available indicator and/or control 100 (FIG. 14) to display the temperature reading(s) and, where appropriate, exercise control over the temperature of fluids used to irrigate.

Prior to using cannula canister 20 a conventionally available IV-type tube 102 (FIGS. 2-5 and 12 and 13) of selected length is attached to fluid port 40 (FIGS. 4 and 8) of end piece 24. Alternatively tube 102, of a predetermined length, and temperature probe 70, positioned in accessory port 44 as described above, may be provided in a sterile kit or pack (not shown) for future availability and use.

When used to irrigate (lavage) a patient 120 (FIG.14) one end of tube 102 is to be attached to a conventionally available pump 122, which may be disposed on a conventionally available pole 124, and the other end of tube 102 is connected to port 40 of cannula 20 . Cannula 20, after being so prepared, may then be inserted through a surgical opening, such as 130 of the anatomical body of patient 120, and the irrigation procedure may thereafter proceed. During the irrigation procedure thermocouple 70 may, or may not be, utilized to monitor, the temperature of the fluid passing through cannula 20. Such monitored temperature may, in turn, be transmitted to indicator or control 100 which may be utilized to provide an appropriate indication to the person(s) administrating the respective procedure and/or may effect a pre-set control over the fluid being utilized.

When used to aspirate (suction) patient 120 (FIG. FIGS. 12 and 14) one end of tube 102 is to be attached to a conventionally available suction fitting or device 130 (FIG. 14), conventionally available at most medical facilities, and the other end of tube 102 connected to port 40 of cannula 20 . Cannula 20, after being so prepared, may then be inserted through the surgical opening, such as 130 of the anatomical body of patient 120, and the aspiration (suctioning) procedure may thereafter proceed. During the aspiration (suctioning) procedure thermocouple 70 may, or may not be, utilized to monitor the temperature of the fluid passing through cannula 20. Such monitored temperature may, in turn, be transmitted to indicator or control 100 which may be utilized to provide an appropriate indication to the person(s) administrating the respective procedure and/or may effect a pre-set control over the aspirating procedure.

1. A cannula; comprising,
(a) a perforated, tubular canister wall of selected length, cross-section configuration, and wall thickness, having a first end cap at a first end and a second end cap at a second end; and
(b) said first end cap further including a fluid port of predetermined size and configuration and a temperature port of predetermined size and configuration.

2. The cannula of claim 1 wherein said cross-section configuration of said tubular canister is substantially circular.

3. The cannula of claim 1 wherein said perforations extend through no more then 40% of said canister wall and no less then 10% of said canister wall.

4. The cannula of claim 1 wherein said perforations are substantially rectangular with rounded corners, are substantially 0.050 inches by 0.090 inches in size and extend through substantially 14% of said canister wall.

5. The cannula of claim 1 wherein said canister wall, and said end caps, are fabricated from biocompatible thermoplastics, including but not limited to, polyethylene, polypropylene, polyester, nylon, polyvinyl chloride, acrylonitrile butadiene styrene, polytetrafluoroethylene, acrylic, polycarbonate; and are compatible with various sterilization processes, including but not limited to, ethylene oxide gas and gamma-radiation.

6. The cannula of claim 1 wherein said end caps are joined to said canister wall by a variety of fabrication techniques including, but not limited to, adhesive bonding, solvent bonding, ultrasonic welding, RF welding, direct heat, spin welding, snap interference fit etc.

7. The cannula of claim 1 wherein said fluid port of said first end cap includes a first part that extends above a surface wall of said first end cap and a second part that extends below said surface wall of said first end cap and wherein at least said first part is sized and configured to have attached thereto IV-type tubing of predetermined length.

8. The cannula of claim 7 wherein said second part of said fluid port is formed to have an open slot sized and disposed to direct fluid passing through said fluid port towards said temperature port.

9. The cannula of claim 8 wherein said temperature port is sized, and configured to receive a sensing end of a temperature probe or sensor and to position such sensing end in the path of fluid passing through the cannula.

10. The cannula of claim 9 wherein the temperature probe or sensor may have a connecting end to connect it to a device to display and react to fluid temperature sensed.

11. The cannula of claim 1 wherein said canister is sized and configured for use in either an aspiration (suctioning) procedure or an irrigation (lavage) procedure.

12. The cannula of claim 11 wherein a pump may be attached to said fluid port and to a source of appropriate fluid to provide a flow of fluid into and through said canister.

13. The cannula of claim 12 wherein the fluid flow is to be directed into an anatomical body of a person.

14. The cannula of claim 13 wherein a temperature probe or sensor is attached to said canister so that at least a sensing portion of the temperature or sensor is disposed within the path of fluid-flow through said canister.

15. The cannula of claim 12 wherein a suctioning device is attached to said fluid port to withdraw fluid from an anatomical body of a person.

16. The cannula of claim 13 wherein a thermocouple is attached to said canister so that at least a sensing portion of the thermocouple is disposed within the path of fluid flow through said canister.

17. The cannula of claim 1 wherein said second end cap also includes perforations extending through a wall of said second end cap.

18. The cannula of claim 1 wherein said first end cap and said second end cap each include a circumferential channel within which an end portion of said tubular canister wall is secured to so form said canister.

19. (canceled)

20. The cannula of claim 21; further comprising fluid entry means to permit the flow of fluid into and out from said canister means; and temperature positioning means to facilitate positioning of a temperature probe means within the path of fluid flow through said canister means.

21. A cannula; comprising,
(a) canister means to facilitate patient treatments;
(b) canister wall means of selected length, cross-section configuration, and thickness to facilitate fabrication of said cannula;
(c) first end cap means for said canister means secured to a first end of said canister wall means to fabricate said cannister means;
(d) second end cap means for said canister means secured to a second end of said canister wall means to fabricate said cannister;
(e) fluid port means of predetermined size and configuration to facilitate fluid flow through said canister means;
(f) thermoprobe port means of predetermined size and configuration provided for said canister means. and
(g) opening means extending through approximately 14% of said canister means.
